(19) United States
(12) Patent Application Publication
(10) Pub. No.: US 2021/0290604 A1
(43) Pub. Date: Sep. 23, 2021

(54) COMPOUNDS FOR TREATMENT OF INFLAMMATORY BOWEL DISEASE AND METHODS THEREOF

(71) Applicant: ALGERNON PHARMACEUTICALS INC., Vancouver (CA)

(72) Inventor: Mark WILLIAMS, Winnipeg (CA)

(21) Appl. No.: 17/258,393

(22) PCT Filed: Jul. 3, 2019
(86) PCT No.: PCT/CA2019/050913

§ 371 Date: Jan. 6, 2021

Related U.S. Application Data
(60) Provisional application No. 62/809,363, filed on Feb. 22, 2019.
(60) Provisional application No. 62/778,744, filed on Dec. 12, 2018.
(60) Provisional application No. 62/694,898, filed on Jul. 6, 2018.

(51) Int. Cl. A61K 31/44; A61K 45/06; A61K 31/4709; A61K 31/4164

(52) U.S. Cl. CPC A61K 31/44; A61K 31/4164; A61K 31/4709; A61K 45/06

(57) ABSTRACT

The current application relates to the use of emoxypine or a glutamate 2b receptor (Glut2B) antagonist for the treatment or prophylaxis of colitis or inflammatory bowel disease (IBD) in a subject. The glut2B antagonist or emoxypine can also be combined with other drugs such as anti-inflammatory drugs, immune system suppressors, antibiotics, anti-diarrheas, pain relievers, iron supplements, vitamin B12, or calcium/vitamin D supplements.

### REFERENCE TO RELATED APPLICATIONS

The present application claims priority from U.S. provisional application No. 62/694,898 filed Jul. 6, 2018, U.S. provisional application No. 62/778,744 filed Dec. 12, 2018 and U.S. provisional application No. 62/809,363 filed Feb. 22, 2019, the contents of which are hereby incorporated by reference.

### FIELD OF INVENTION

The present invention relates to the use of compounds for treating inflammatory bowel disease, and in particular, the use of glutamate 2b receptor antagonists, and/or emoxypine, for treating inflammatory bowel disease, ulcerative colitis (UC), and Crohn's Disease.

### BACKGROUND

Inflammatory Bowel Disease (IBD) is characterized by an inflamed colon and/or small intestine. Most commonly, IBD is either Crohn's Disease or UC. IBD also includes indeterminate colitis. Indeterminate colitis is a term used when it is unclear if the inflammation is due to Crohn's disease or ulcerative colitis.

Ulcerative Colitis effects the colon only, and is marked by showing no healthy areas in the colon.

Crohn's disease is a type of inflammatory bowel disease (IBD) that primarily involves the small and large intestine, but may also affect any other part of the gastrointestinal tract. In its mild forms, Crohn's disease causes scattered, shallow ulcers in the inner surface of the bowel. In more serious cases, deeper and larger ulcers can develop, causing scarring and stiffness and possibly narrowing of the bowel, sometimes leading to obstruction. Deep ulcers can puncture holes in the bowel wall, leading to infection in the abdominal cavity (peritonitis) and in adjacent organs.

Common symptoms of IBD disease include abdominal pain, diarrhea, vomiting, fever, and weight loss. While the causes of IBD are unknown, genetic, environmental, and lifestyle factors are understood to contribute to IBD.

There is currently no cure for IBD, and there is no single treatment that works for every individual. 5-aminosalicylic acid, also known as 5-ASA, is currently a global standard for treatment of IBD. Used in combination with other drugs, the goal of such treatment is to reduce the inflammation that triggers the individual's signs and symptoms, and to improve long-term prognosis by limiting complications.

The murine models of intestinal inflammation are well-characterized experimental models of intestinal immune dysregulation that ultimately lead to colitis—a common characteristic of inflammatory bowel diseases. In these models, the progression of colonic inflammation is highly predictable and reproducible, leading to significant infiltration of the lamina propria with inflammatory cells and tissue damage of the colonic mucosa (Kiesler et al., Experimental models of inflammatory bowel diseases, Cell Mol Gastroenterol Hepatol, 2015; 1:154-70). In one such model, 2,4,6-trinitrobenzene sulfonic acid (TNBS) is used to induce colitis in mice. Ethanol and TNBS are co-administered intra-rectally to rats, as the ethanol is used as a means to effectively disrupt intestinal barrier and enable the interaction of TNBS with colon tissue proteins. (Antoniou et al., The TNBS-induced colitis animal model: An overview, Ann Med Surg (Lond). 2016 November; 11: 9-15.) TNBS-induced colitis in mice constitutes an animal model of ulcerative colitis (UC) with high degree of similarity to the histopathological characteristics and distribution of inflammation described in human ulcerative colitis.

The present invention provides a novel use of existing drugs, typically studied as potential therapies for neurological conditions, for the treatment and/or alleviation of IBD.

### SUMMARY OF INVENTION

In one aspect, the present invention provides methods and uses of Emoxypine for the treatment or prophylaxis of colitis or inflammatory bowel disease (IBD) in a subject.

In an embodiment of the invention, a glutamate 2b receptor (Glut2B) antagonist for the treatment or prophylaxis of colitis or inflammatory bowel disease (IBD) in a subject. The Glut2B antagonist may be one or more of Ifenprodil, Radiprodil, Traxoprodil, Rislenmdaz, Eliprodil, Ro-25, 6981, and BMT-108908.

In another embodiment of the invention, the compounds of the invention are used in combination with the use of one or more of: anti-inflammatory drugs, immune system suppressors, antibiotics, anti-diarrheals, pain relievers, iron supplements, vitamin B-12 shots, and calcium and vitamin D supplements. The anti-inflammatory drugs may be one or more of corticosteroids and oral 5-aminosalicylates. The immune system suppressors may be one or more of azathioprine, mercaptopurine, infliximab, adalimumab, certolizumab pegol and vedolizumab. The antibiotics may be one or more of iprofloxacin and metronidazole.

In a further aspect, the inflammatory bowel disease is Crohn's disease.

In an embodiment of the invention, the inflammatory bowel disease is ulcerative colitis.

### BRIEF DESCRIPTION OF THE FIGURES

Exemplary embodiments are illustrated in referenced figures of the drawings. It is intended that the embodiments and figures disclosed herein are to be considered illustrative rather than restrictive.

FIG. 1 is a line graph comparing the mean percentage change in body weights from baseline (±the standard error of the mean, or SEM) for the experimental treatment groups of mice, test compounds Emoxypine, Ifenprodil and Radiprodil, compared to the “Naïve” control group, the “TNBS-Vehicle” control group, and the “5-ASA” positive control group.

FIG. 2 is a line graph comparing the mean Disease Activity Index (DAI) data, which includes daily measurement of body weight and evaluation of stool consistency, from baseline (±the standard error of the mean, or SEM) for the experimental treatment groups of mice, test compounds Emoxypine, Ifenprodil and Radiprodil, compared to the “Naïve” control group, the “TNBS-Vehicle” control group, and the “5-ASA” positive control group.

FIG. 3 is a line graph comparing the mean fecal consistency from baseline (±the standard error of the mean, or SEM) for the experimental treatment groups of mice, test compounds Emoxypine, Ifenprodil and Radiprodil, compared to the “Naïve” control group, the “TNBS-Vehicle” control group, and the “5-ASA” positive control group.

FIG. 4 is a line graph comparing the mean occult positivity from baseline (±the standard error of the mean, or SEM) for the experimental treatment groups of mice, test compounds Emoxypine, Ifenprodil and Radiprodil, compared to the “Naïve” control group, the “TNBS-Vehicle” control group, and the “5-ASA” positive control group.

FIG. 5 is a column graph showing an indication of disease severity with a comparison of the mean colon length in centimeters for the experimental treatment groups of mice, test compounds Emoxypine, Ifenprodil and Radiprodil, compared to the “Naïve” control group, the “TNBS-Vehicle” control group, and the “5-ASA” positive control group.

FIG. 6 is a column graph showing an indication of disease severity with a comparison of the mean colon weight in grams for the experimental treatment groups of mice, test compounds Emoxypine, Ifenprodil and Radiprodil, compared to the “Naïve” control group, the “TNBS-Vehicle” control group, and the “5-ASA” positive control group.

FIG. 7 is a column graph showing an indication of disease severity with a comparison of the mean colon weight/length ratio in milligrams/centimeter for the experimental treatment groups of mice, test compounds Emoxypine, Ifenprodil and Radiprodil, compared to the “Naïve” control group, the “TNBS-Vehicle” control group, and the “5-ASA” positive control group.

FIG. 8 is a line graph showing an indication of disease progression with a comparison of the daily survival rate in percentage for the experimental treatment groups of mice, test compounds Emoxypine, Ifenprodil and Radiprodil, compared to the “Naïve” control group, the “TNBS-Vehicle” control group, and the “5-ASA” positive control group.

FIG. 9 is a column graph showing a Histopathology severity score, which evaluates of colitis, bowel wall inflammation, leukocyte infiltration, high vascular density, bowel wall thickening, disruption of normal crypt architecture and epithelial ulceration, with a comparison of the Histopathology Scores for the experimental treatment groups of mice, test compounds Emoxypine, Ifenprodil and Radiprodil, compared to the “Naïve” control group, the “TNBS-Vehicle” control group, and the “5-ASA” positive control group.

### DETAILED DESCRIPTION

Throughout the following description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The inventor has found that a number of pharmacologic compounds approved for use in other pathologies are useful in inhibiting or alleviating colitis and may be useful in the prophylaxis and/or treatment of inflammatory bowel disease. In some embodiments, it is found that in the murine model of TNBS-induced colitis, the level of colonic inflammation is inhibited or alleviated. Based on the experimental results described herein, it can be soundly predicted that the compounds described herein will be useful in some embodiments in the prophylaxis and/or treatment of colitis or inflammatory bowel disease.

A currently used therapy for treating ulcerative colitis and inflammatory bowel disease is administering the pharmacologic compound 5-ASA, which was used as a positive control in the experimental examples described herein.

5-ASA (5-Aminosalicylic Acid), 5-Amino-2-hydroxybenzoic acid, is an aminosalicylate anti-inflammatory drug known in the art for treating inflammatory bowel disease such as ulcerative colitis and for maintaining remission in Crohn's disease. The chemical structure of 5-Aminosalicylic Acid is as follows:

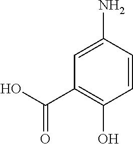 [SMILES: Nc1ccc(O)c(C(=O)O)c1]

The examples and data below show the effects of inhibiting or alleviating colitis by administering a therapeutically effective amount of Emoxypine or glutamate 2b receptor antagonists, in particular, Ifenprodil and Radiprodil. These compounds described herein are existing drugs, typically known for treatment of neurological conditions.

### Use of Emoxypine

Emoxypine, 2-Ethyl-6-methyl-3-hydroxypyridine, is known in the art as an antioxidant. The chemical structure of Emoxypine is as follows:

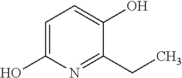

In one aspect, the present invention provides a use and method of treatment or prophylaxis of colitis or inflammatory bowel disease (IBD) in a subject with Emoxypine or a pharmaceutically acceptable salt thereof. The IBD may be Crohn's disease or ulcerative colitis, among others.

In a preferred embodiment, the amount of Emoxypine used is between 0.1 to 30 mg per kg of the subject.

In a further preferred embodiment, the amount of Emoxypine used is between 5 to 20 mg per kg of the subject.

In a still further preferred embodiment, the amount of Emoxypine used is about 13 mg per kg of the subject.

The Emoxypine, or pharmaceutically acceptable salt thereof, may be administered to the subject orally, intravenously or in a manner known in the art. The Emoxypine, or pharmaceutically acceptable salt thereof, may also be administered with one or more pharmaceutically acceptable excipients.

### Use of Glutamate 26 Receptor Antagonists

Glutamate 2b receptor antagonists, a category of glutamate NMDA receptor antagonists, work to inhibit the action of the N-methyl-d-aspartate receptor (the NMDA receptor). Studies have focused on their application in neurological disorders, such as depression, Parkinson's disease, epilepsy, Huntington's disease, neuropathic pain, traumatic brain injury and stroke. The generic structure of selective NR2B antagonists consists of a central tertiary amine, linked to a (substituted) aromatic ring on two sides as shown below.

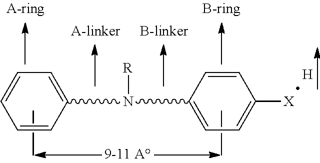

As noted in Nikam et al., NR2B Selective NMDA Receptor Antagonists, Current Pharmaceutical Design, 2002, 8, 845-855, functional NMDA receptors are composed of different combinations of multiple protein subunits. Five distinct gene products, NR1, NR2A, NR2B, NR2C and NR2D, are all expressed in the mammalian CNS. There are 8 isoforms of the NR1 subunit, due to differential splicing of three inserts. The physiological receptor is a heteromer containing an NR1 subunit with one or more of the different NR2 subunits. In in vitro experiments, the NR2 subunit that is coupled to the NR1 subunit alters the electrophysiological and pharmacological properties of the formed receptor channel. Additionally, activation of the NMDA receptor has an absolute requirement for glycine as a co-agonist.

This differential receptor distribution presents the possibility that compounds selective for an individual NR2 subunit may possess some of the therapeutic properties of the broad spectrum NMDA antagonists but lack their side effect profile.

Given the similar chemical structures of various types of glutamate 2b receptor antagonists described below, it can be soundly predicted that various forms of glutamate 2b receptor antagonists will produce a similar biological response as that of Ifenprodil and Radiprodil, and thereby will be useful in some embodiments in the prophylaxis and/or treatment of colitis or inflammatory bowel disease.

Examples of glutamate 2b receptor antagonists are: Ifenprodil, Eliprodil, Radiprodil, Traxoprodil, Rislenemdaz, Ro-25, 6981, and BMT-108908.

Ifenprodil, 4-[2-(4-benzylpiperidin-1-ium-1-yl)-1-hydroxypropyl] phenol; 2,3,4-trihydroxy-4-oxobutanoate, is known in the art as a selective NMDA receptor (glutamate) antagonist. Ifenprodil was originally (in the early 1970's) developed as a vasodilator. Ifenprodil is currently being studied for treatment of adolescent PTSD. The chemical structure is as follows:

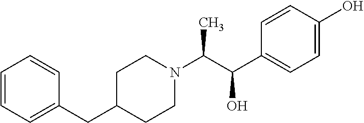 [SMILES: C[C@@H]([C@H](O)c1ccc(O)cc1)N1CCC(Cc2ccccc2)CC1]

In some embodiments tested in the examples herein, Ifenprodil hemitartrate having the following structure was used:

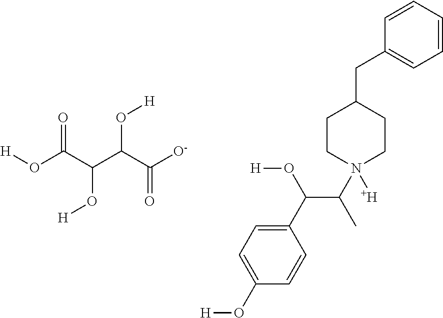

Eliprodil, (4-chlorophenyl)-4-((4-fluorophenyl)methyl)-1-piperidineethanol, is known in the art as an NMDA receptor antagonist developed to have better oral bioavailability than Ifenprodil. It is currently being studied for therapeutic treatment of Parkinson's disease. The chemical structure of Eliprodil is as follows:

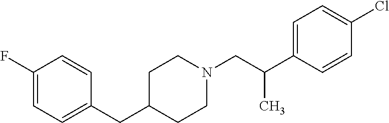 [SMILES: CC(CN1CCC(Cc2ccc(F)cc2)CC1)c1ccc(Cl)cc1]

Radiprodil, 2-[4-[(4-fluorophenyl)methyl]piperidin-1-yl]-2-oxo-N-(2-oxo-3H-1,3-benzoxazol-6-yl)acetamide, is known in the art as an NMDA receptor antagonist. It has been used in trials studying the treatment of Infantile Spasms (IS) and Diabetic Peripheral Neuropathic Pain. The chemical structure of Radiprodil is as follows:

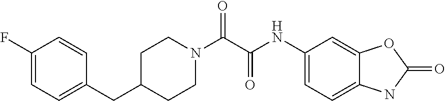

Traxoprodil, 1-((1S,2S)-1-hydroxy-1-(4-hydroxyphenyl)propan-2-yl)-4-phenylpiperidin-4-ol, is known in the art as an NMDAR antagonist with unique NR2B specificity, potentially useful in treating Parkinson's disease, stroke, and major depressive disorders. The chemical structure of Traxoprodil is as follows:

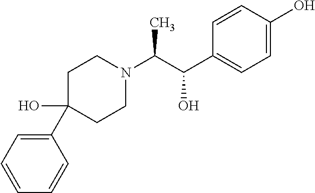 [SMILES: C[C@@H]([C@@H](O)c1ccc(O)cc1)N1CCC(O)(c2ccccc2)CC1]

Rislenmdaz, 4-methylbenzyl 3-fluoro-4-((pyrimidin-2-ylamino)methyl)piperidine-1-carboxylate, is known in the art as a selective NMDA receptor (glutamate) antagonist associated with potential therapeutic treatments of schizophrenia and depression. The chemical structure of Rislenmdaz is as follows:

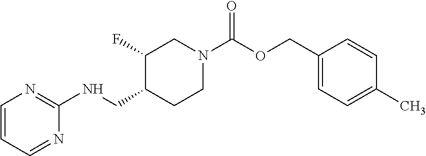 [SMILES: Cc1ccc(COC(=O)N2CC[C@H](CNc3ncccn3)[C@H](F)C2)cc1]

Ro 25-6981 is known in art as a glutamate 2B receptor antagonist with the following chemical structure:

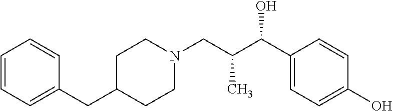 [SMILES: C[C@H](CN1CCC(Cc2ccccc2)CC1)[C@H](O)c1ccc(O)cc1]

BMT-108908 is known in art as a glutamate 2B receptor antagonist with the following chemical structure:

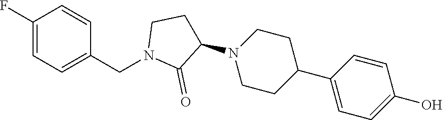 [SMILES: O=C1[C@H](N2CCC(c3ccc(O)cc3)CC2)CCN1Cc1ccc(F)cc1]

In another aspect, the present invention provides a use and method of treatment or prophylaxis of colitis or inflammatory bowel disease (IBD) in a subject with glutamate 2b receptor antagonists. The IBD may be Crohn's disease or ulcerative colitis, among others.

In a preferred embodiment, the amount of glutamate 2b receptor antagonist used is between 0.2 and 40 mg per kg, preferably between 0.5 to 10 mg per kg of the subject.

In a further preferred embodiment, the amount of glutamate 2b receptor antagonist used is between 1 to 5 mg per kg of the subject.

In a still further preferred embodiment, the amount of glutamate 2b receptor antagonist used is abut 2.5 mg per kg of the subject.

In an embodiment, the glutamate 2b receptor antagonists may be Ifenprodil, Eliprodil, Radiprodil, Traxoprodil, Rislenmdaz, Ro-25, 6981, or BMT-108908.

The glutamate 2b receptor antagonists may be administered to the subject orally, intravenously or in any manner known in the art. The glutamate 2b receptor antagonists may also be administered with one or more pharmaceutically acceptable excipients.

### Use in Combination

In another aspect, the present invention provides a use and method of treatment or prophylaxis of colitis or inflammatory bowel disease in a subject with Emoxypine or glutamate 2b receptor antagonists in combination with one or more of: anti-inflammatory drugs, immune system suppressors, antibiotics, anti-diarrheals, pain relievers, iron supplements, vitamin B-12 shots, and calcium and vitamin D supplements.

The anti-inflammatory drugs are often used in the first step in the treatment of inflammatory bowel disease. They include corticosteroids and/or oral 5-aminosalicylates.

Oral 5-aminosalicylates include sulfasalazine (for example Azulfidine), which contains sulfa, and mesalamine (for example Asacol HD, Delzicol or others). While oral 5-aminosalicylates have been widely used in the past to treat Crohn's disease, it is now generally considered of limited benefit.

Corticosteroids, such as prednisone and budesonide (for example Entocort EC), may help reduce inflammation in the body, though it has not been shown to work for everyone with Crohn's disease. It is prescribed, typically, only if the subject hasn't responded to other treatments. Corticosteroids may be used for short-term (three to four months) symptom improvement and to induce remission. Corticosteroids may also be used in combination with an immune system suppressor.

Immune system suppressor also reduce inflammation in a subject, but they target the subject's immune system, which produces the substances that cause inflammation. For some, a combination of these drugs works better than one drug alone. Such immunosuppressant drugs used with Emoxypine or glutamate 2b receptor antagonists may comprise one or more of azathioprine, mercaptopurine, infliximab, adalimumab, certolizumab pegol and vedolizumab.

Azathioprine (for example Azasan or Imuran) and mercaptopurine (for example Purinethol or Purixan) are the most widely used immunosuppressants for treatment of inflammatory bowel disease. As they tend to lower resistance to infection and inflammation of the liver, taking them requires a subject to be followed up closely with a doctor and to have his/her blood checked regularly to look for such side effects. Their use may also cause nausea and vomiting.

Infliximab (for example Remicade), adalimumab (for example Humira) and certolizumab pegol (for example Cimzia) are called TNF inhibitors or biologics. They work by neutralizing an immune system protein known as tumor necrosis factor (TNF).

Vedolizumab was recently approved for use in treatment of Crohn's disease. Vedolizumab works like natalizumab, but appears not to carry a risk of brain disease.

Antibiotics have been used in the past to reduce the amount of drainage and sometimes heal fistulas and abscesses in subjects with Crohn's disease. Some researchers also think antibiotics may help reduce harmful intestinal bacteria that may play a role in activating the intestinal immune system, leading to inflammation. Frequently prescribed antibiotics include ciprofloxacin (for example Cipro) and metronidazole (for example Flagyl).

In addition to controlling inflammation, Emoxypine or glutamate 2b receptor antagonists may also be used with other medications described below to help relieve other signs and symptoms.

Anti-diarrheals includes fiber supplements, such as psyllium powder (for example Metamucil) or methylcellulose (for example Citrucel), may be used to help relieve mild to moderate diarrhea by adding bulk to stool. For more severe diarrhea, loperamide (for example Imodium A-D) may be used.

Pain relievers, for mild pain, such as acetaminophen (for example Tylenol or others) may be used. However, other common pain relievers, such as ibuprofen (for example Advil, Motrin IB and others) and naproxen sodium (for example Aleve) should not be used as these drugs tend to make a subject's symptoms worse, and can make the disease worse as well.

If the subject has chronic intestinal bleeding, the subject may develop iron deficiency anemia and may need to take iron supplements.

Crohn's disease can cause vitamin B-12 deficiency. As such, Vitamin B-12 shots may be used with Emoxypine or glutamate 2b receptor antagonists to help prevent anemia, promote normal growth and development, since Vitamin B-12 s is essential for proper nerve function.

Calcium and vitamin D supplements may also be used with Emoxypine. Crohn's disease and the steroids used to treat it can increase a subject's risk of osteoporosis. Calcium supplement with added vitamin D may help alleviate this osteoporosis.

Embodiments of the present invention are further described with reference to the following examples, which are intended to be illustrative and not limiting in nature.

### Example—Materials and Methods

The test compounds Emoxypine and Ifenprodil were obtained from Toronto Research Chemicals, Toronto, ON, Canada M3J2K8. Radiprodil was obtained from Axon Medchem LLC, Mclean, Va. 22102, USA. The Positive control 5-aminosalicylic acid (5-ASA) was obtained from Sigma Aldrich, USA. The vehicle used was 0.5% CMC.

The dose selected for the animal studies was determined by taking the maximum known human daily dose, dividing by the average weight if an adult (˜60-70 kg) to get a human mg/kg dose. Then that number was multiplied by 12 to convert to a mouse dose based on conventional dosing tables. See Nair and Jacob, J Basic Clin Pharm March 2016-May 2016, 7(2):27-31.

Thus, working backwards to arrive at the human dose, as examples:

Emoxypine=160 mg/kg divide 12=13.3 mg/kg,

Ifenprodil=30 mg/kg divide 12=2.5 mg/kg,

Radiprodil=30 mg/kg divide 12=2.5 mg/kg.

Healthy young female SJL mice were used for the study. At the commencement of the study, the mice were between 8-9 weeks of age, weighing 20-22 g. All the mice were obtained from The Jackson Laboratory, Bar Harbor, Me. 04609 USA.

The mice were maintained in a controlled environment with a temperature of 70-72° F., humidity 30-70%, with a photo cycle of 12 hours of light and 12 hours of dark. They were provided with TEKLAD 2018-Global 18% diet and Arrowhead drinking water ad libitium.

After seven days of acclimatization, mice were grouped according to their body weight. There was one group of ten mice and five groups of fifteen mice each. Five groups of fifteen mice each were challenged intra-rectally with 100 μl of 2.0% TNBS in 50% EtOH and other group of ten mice were challenged intra-rectally with 100 μl of 50% EtOH and serve as No-TNBS control. The experimental groups were as follows:

TABLE 1
Experimental Design

| | | | | Dose | Dosing | Dosing |
|---|---|---|---|---|---|---|
| Group | Description | N | ROA | mg/kg | Volume | Frequency |
| 1 | Naive | 10 | PO | xxxx | 10 ml/kg | QD |
| | (100 ul of | | | | | Days 1-7 |
| | 50% EtOH | | | | | |
| | intrarectal- | | | | | |
| | once) | | | | | |
| 2 | Vehicle | 15 | PO | xxxx | 10 ml/kg | QD |
| | (100 ul of | | | | | Days 1-7 |
| | 2.0% TNBS | | | | | |
| | in50% EtOH | | | | | |
| | intrarectal- | | | | | |
| | once) | | | | | |
| 3 | 5-ASA | 15 | PO | 100 | 10 ml/kg | QD |
| | (100 ul of | | | | | Days 1-7 |
| | 2.0% TNBS | | | | | |
| | in 50% EtOH | | | | | |
| | intrarectal- | | | | | |
| | once) | | | | | |
| 4 | Emoxypine | 15 | PO | 160 | 10 ml/kg | QD |
| | (100 ul of | | | | | Days 1-7 |
| | 2.0% TNBS | | | | | |
| | in 50% EtOH | | | | | |
| | intrarectal- | | | | | |
| | once) | | | | | |
| 5 | Ifenprodil | 15 | PO | 30 | 10 ml/kg | QD |
| | (100 ul of | | | | | Days 1-7 |
| | 2.0% TNBS | | | | | |
| | in 50% EtOH | | | | | |
| | intrarectal- | | | | | |
| | once) | | | | | |
| 6 | Radiprodil | 15 | PO | 30 | 10 ml/kg | QD |
| | (100 ul of | | | | | Days 1-7 |
| | 2.0% TNBS | | | | | |
| | in 50% EtOH | | | | | |
| | intrarectal- | | | | | |
| | once) | | | | | |

The mice were challenged intra-rectally with 100 μl of 2% TNBS in 50% ethanol under light anesthesia with ketamine/xylazine. The test compounds were administrated an hour prior to intra-rectal administration of TNBS as per scheduled daily dosing.

The test compounds, Emoxypine, Ifenprodil and Radiprodil were prepared in 0.5% CMC and administrated orally once-a-day from day 1 to 7. 5-ASA was also prepared in 0.5% CMC and administrated orally once a day beginning on Day 1 to Day 7. Vehicle and no-TNBS control groups received 0.5% CMC orally from Day 1 to Day 7.

The following measurements and assessments were taken for each mouse.

Body Weight: The body weights were measured daily for 1-7 days using a laboratory balance.

Disease Activity Index: The clinical assessment of the mice was performed beginning Day 2 (a day after the intra-rectal administration of TNBS). The clinical assessment includes body weight, stool consistency and the presence of blood in the stools and scored according to Table 2.

TABLE 2
Disease Activity Index

| | Weight | Stool | Occult/Gross |
|---|---|---|---|
| Score | Loss (%) | Consistency | bleeding |
| 0 | No Loss | Normal | Normal |
| 1 | 1-5 | | |
| 2 | 5-10 | Loose | Occult |
| 3 | 10-15 | | |
| 4 | >15 | Diarrhea | Gross Bleeding |

Serum Collection: on Day 8, blood samples were collected from all the surviving mice and were processed for serum and stored at −80° C.

Colon Length and Weight: the mice were euthanized using CO2 asphyxiation and colon from the colocecal junction to the anus was removed, washed and cleaned of all fecal matter using PBS. The colon length and weight were measured and then preserved in 10% NBF for histopathology.

Histopathology: Formalin fixed colon samples were submitted to affiliated histopathology laboratory for histopathological analysis subjected to hematoxylin and eosin (H & E) staining using standard techniques. Each colon was trimmed from both ends and mid. All three sections were stained and evaluated.

A board certified veterinarian pathologist assessed the presence of colitis and severity score according to the following criteria:
0=no sign of inflammation
1=very low level of inflammation
2=low level of leukocytic infiltration, low level of inflammation
3=high level of leukocytic infiltration, high vascular density, inflammation and thickening of colon wall
4=transmural infiltration, loss of goblet cells, high vascular density, crypt abscesses, thickening of colon wall and ulceration.

The data is presented as the mean±standard error (SEM) obtained from Microsoft Excel or GraphPad Prism version 5.00 for Windows (GraphPad Software, San Diego Calif. USA). The data was analyzed using two-way ANOVA using Bonferroni post-test. Differences between groups were considered significant at p<0.05.

### Results

Body Weight

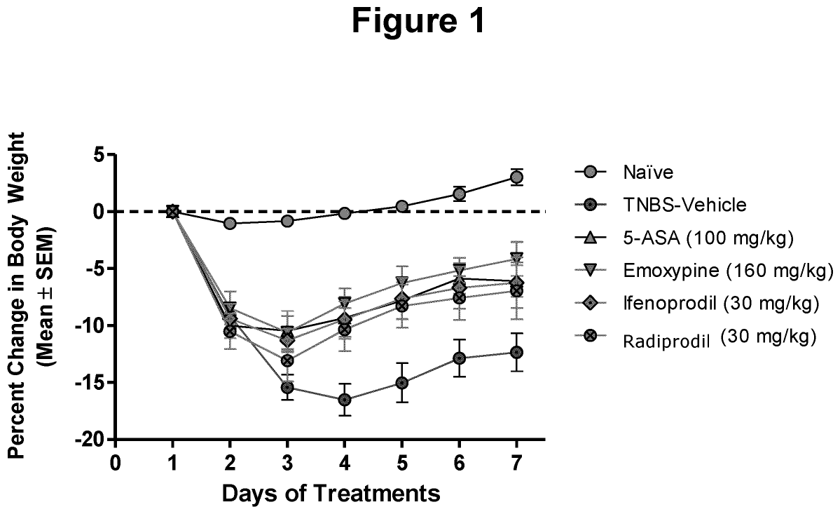

The percentage changes in body weights are summarized in FIG. 1 and Table 3. The decrease in body weight gains were observed from Day 3 till Day 5 and then started recovering. Significant differences were observed with the groups treated with Emoxypine (160 mg/kg po) and Ifenprodil (30 mg/kg po) and 5-ASA (100 mg/kg po). They showed significant improvement beginning on Day 4 as compared to the TNBS-vehicle group. Radiprodil (30 mg/kg po) also showed significant improvement beginning Day 4, with the exception of Day 3 as compared to the TNBS-vehicle group.

Disease Activity Index

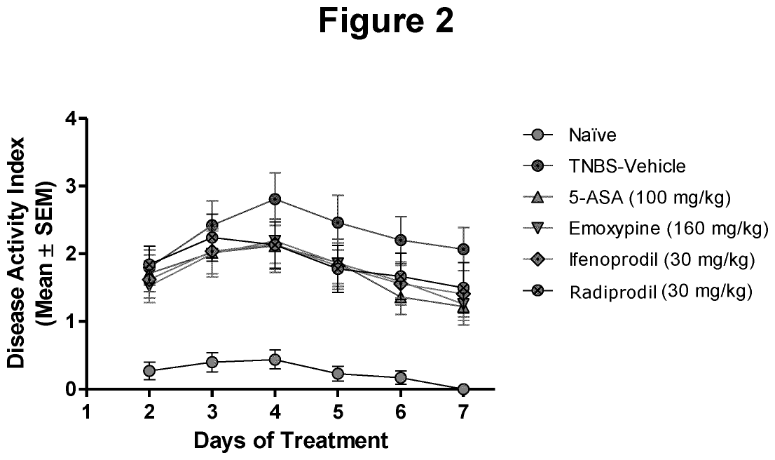

The Disease Activity Index (DAI) data are summarized in FIG. 2 and Table 4. The DAI included daily measurement of body weight and evaluation of stool consistency. No significant differences were observed between treatment groups and TNBS-vehicle group though the response was better with Emoxypine (160 mg/kg), followed by Ifenprodil (30 mg/kg), 5-ASA (100 mg/kg) and Radiprodil (30 mg/kg) treated groups.

Fecal Consistency and Occult Positivity

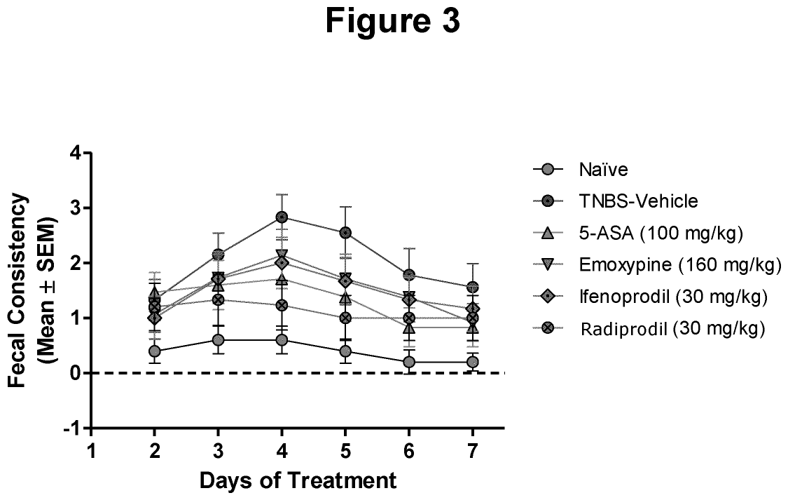

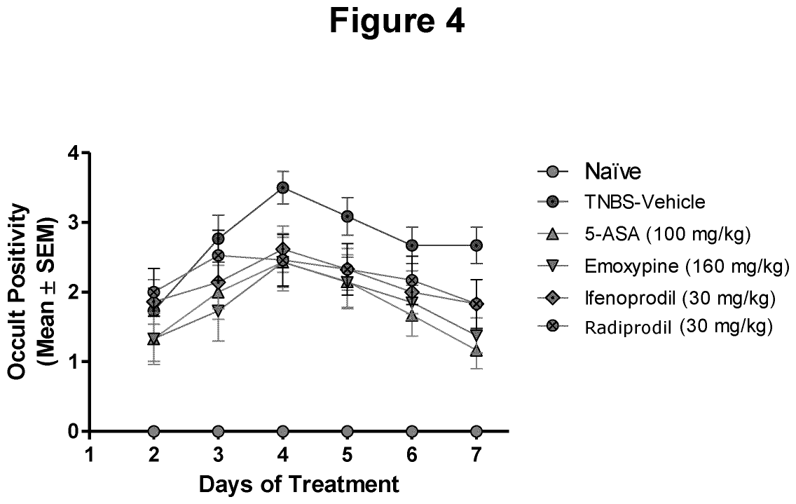

This data is summarized in FIGS. 3-4 and Tables 5-6. No significant differences were observed in fecal consistency between treatment groups and TNBS-vehicle group, whereas significant improvements were observed in occult positivity with the groups treated with Emoxypine (160 mg/kg) and 5-ASA (100 mg/kg on Day 7 as compared to TNBS-vehicle.

Colon Length, Weight and Weight/Length Ratio

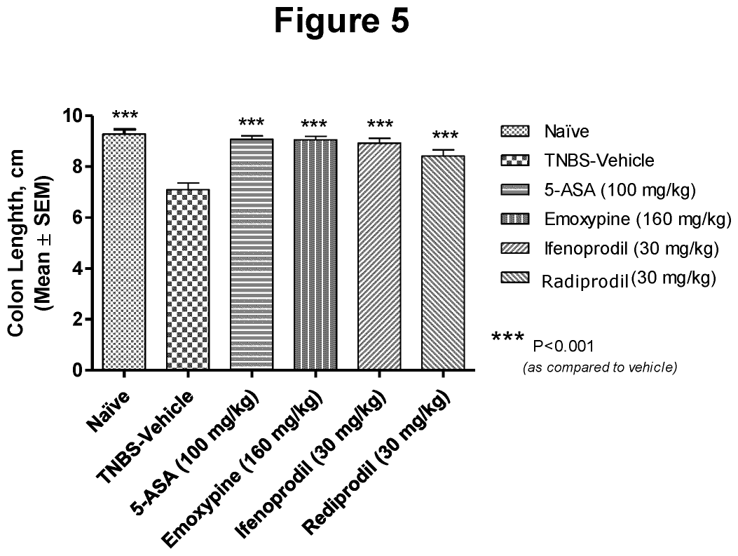

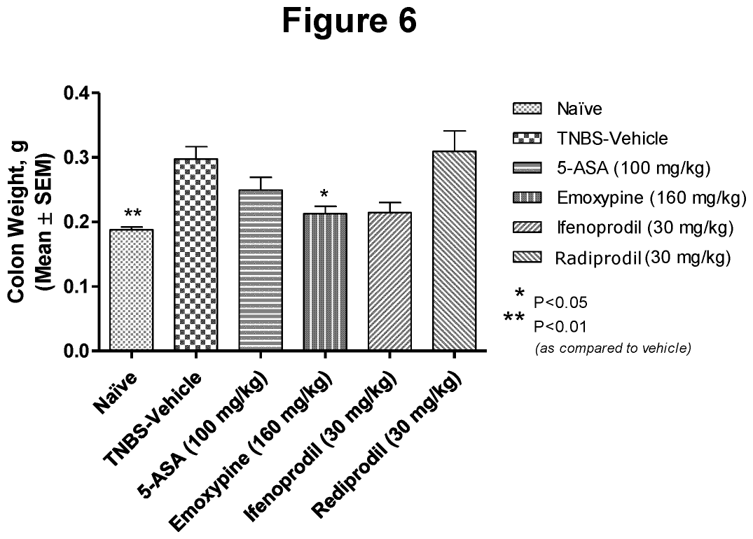

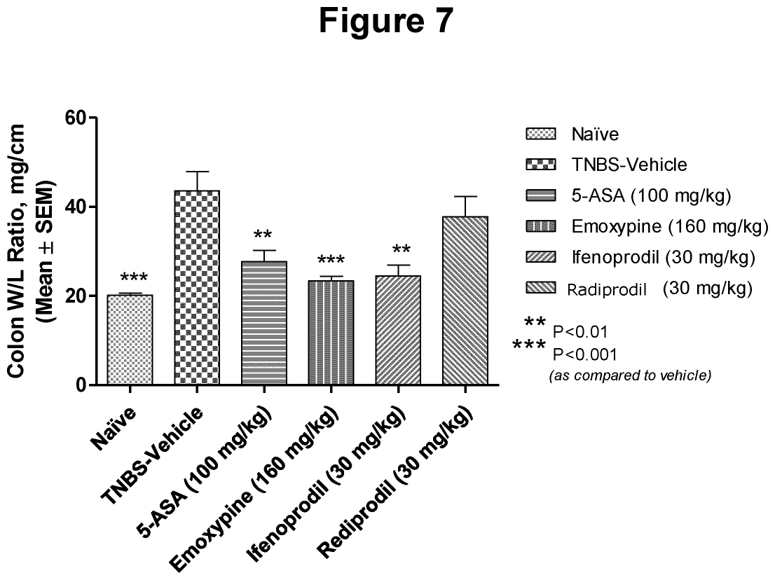

This data is summarized in FIGS. 5-7 and Tables 7-9. Significant differences were observed in colon length between treatments groups and vehicle treated group, whereas in colon weight Emoxypine (160 mg/kg) showed significant improvement as compared to TNBS-vehicle. The ratio between colon weight and length were significant in all the treatment groups as compared to TNBS-vehicle.

Percent Survival

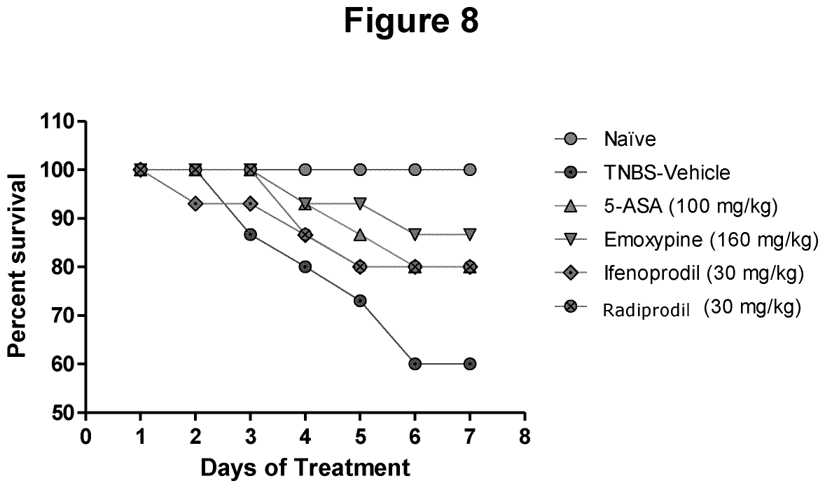

The percent survival data is summarized in FIG. 8 and Table 10. The percent survival was higher with the treatment group treated with Emoxypine (160 mg/kg) followed by Ifenorpdil (30 mg/kg), 5-ASA (100 mg/kg) and Radiprodil (30 mg/kg).

Histopathology

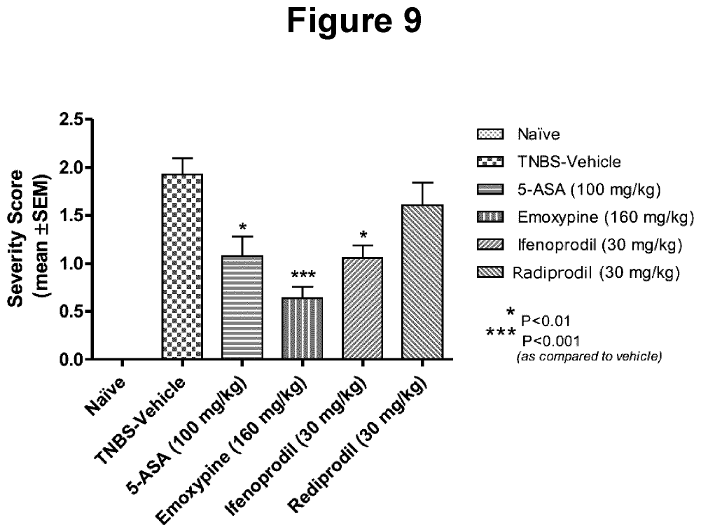

Rating data are summarized in FIG. 9 and Table 11. The data demonstrate that Emoypine and Ifenprodil, like 5-ASA, were statistically significantly reducing cell morphological changes associated with disease. Radiprodil also reduced severity. The 3 main categories include (i) quality and dimension of inflammatory cell infiltrates, (ii) epithelial changes and (iii) overall mucosal architecture.

Detailed statistical analysis of the above data was carried out. Statistical significance is indicated in the Figures.

### CONCLUSIONS

In conclusion, oral administration of Emoxypine at 160 mg/kg, Ifenproodil and Radiprodil at 30 mg/kg showed improvement in colitis as well as in the loss of body weight, DAI, colon length, weight and weight/length ratio as compared to TNBS-vehicle, but the improvement was better with the group treated with Emoxypine followed by Ifenprodil and Radiprodil.

Oral administration of 5-ASA at 100 mg/kg also showed improvement in the colitis, loss of body weight and disease activity index as compared to TNBS-vehicle.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

TABLE 3
Percent Change in Body Weight

| | | | | | | | 5-ASA | | | Emoxypine | | | Ifenprodil | | | Radiprodil | | |
|---|---|---|---|---|---|---|---|---|---|---|---|---|---|---|---|---|---|---|
| | Naïve | | | TNBS-Vehicle | | | (100 mg/kg) | | | (160 mg/kg) | | | (30 mg/kg) | | | (30 mg/kg) | | |
| Days | Mean | SEM | N | Mean | SEM | N | Mean | SEM | N | Mean | SEM | N | Mean | SEM | N | Mean | SEM | N |
| 1. | 0.000 | 0.00 | 10 | 0.00 | 0.00 | 15 | 0.00 | 0.00 | 15 | 0.00 | 0.00 | 15 | 0.00 | 0.0000 | 15 | 0.00 | 0.00 | 15 |
| 2. | −1.030 | 0.44 | 10 | −9.16 | 1.09 | 15 | −10.01 | 1.10 | 15 | −8.46 | 1.44 | 15 | −9.38 | 1.2800 | 15 | −10.51 | 1.56 | 15 |
| 3. | −0.830 | 0.39 | 10 | −15.42 | 1.11 | 15 | −10.45 | 1.74 | 15 | −10.60 | 1.45 | 15 | −11.31 | 1.0100 | 15 | −13.08 | 1.83 | 15 |
| 4. | −0.160 | 0.48 | 10 | −16.51 | 1.40 | 15 | −9.33 | 1.83 | 15 | −8.07 | 1.32 | 15 | −9.44 | 1.5600 | 15 | −10.35 | 1.88 | 15 |
| 5. | 0.460 | 0.44 | 10 | −15.03 | 1.72 | 15 | −7.81 | 1.62 | 15 | −6.26 | 1.46 | 15 | −7.64 | 1.8100 | 15 | −8.29 | 1.89 | 15 |
| 6. | 1.550 | 0.63 | 10 | −12.87 | 1.62 | 15 | −5.87 | 1.33 | 15 | −5.15 | 1.09 | 15 | −6.68 | 1.8600 | 15 | −7.58 | 1.93 | 15 |
| 7. | 3.030 | 0.71 | 10 | −12.34 | 1.67 | 15 | −6.09 | 1.38 | 15 | −4.14 | 1.49 | 15 | −6.23 | 2.2200 | 15 | −6.95 | 2.50 | 15 |

TABLE 4
Disease Activity Index

| | | | | | | | 5-ASA | | | Emoxypine | | | Ifenprodil | | | Radiprodil | | |
|---|---|---|---|---|---|---|---|---|---|---|---|---|---|---|---|---|---|---|
| | Naïve | | | TNBS-Vehicle | | | (100 mg/kg) | | | (160 mg/kg) | | | (30 mg/kg) | | | (30 mg/kg) | | |
| Days | Mean | SEM | N | Mean | SEM | N | Mean | SEM | N | Mean | SEM | N | Mean | SEM | N | Mean | SEM | N |
| 2. | 0.27 | 0.13 | 10 | 1.80 | 0.26 | 15 | 1.71 | 0.27 | 15 | 1.53 | 0.25 | 15 | 1.62 | 0.2700 | 15 | 1.84 | 0.27 | 15 |
| 3. | 0.40 | 0.14 | 10 | 2.42 | 0.36 | 15 | 2.02 | 0.36 | 15 | 2.00 | 0.34 | 15 | 2.04 | 0.3300 | 15 | 2.24 | 0.35 | 15 |
| 4. | 0.44 | 0.14 | 10 | 2.81 | 0.39 | 15 | 2.12 | 0.39 | 15 | 2.19 | 0.33 | 15 | 2.14 | 0.3700 | 15 | 2.13 | 0.34 | 15 |
| 5. | 0.23 | 0.11 | 10 | 2.46 | 0.40 | 15 | 1.87 | 0.35 | 15 | 1.86 | 0.30 | 15 | 1.82 | 0.3400 | 15 | 1.78 | 0.35 | 15 |
| 6. | 0.17 | 0.10 | 10 | 2.20 | 0.35 | 15 | 1.36 | 0.26 | 15 | 1.59 | 0.24 | 15 | 1.56 | 0.3200 | 15 | 1.67 | 0.34 | 15 |
| 7. | 0.00 | 0.00 | 10 | 2.07 | 0.32 | 15 | 1.22 | 0.27 | 15 | 1.26 | 0.24 | 15 | 1.41 | 0.3400 | 15 | 1.50 | 0.37 | 15 |

TABLE 5
Fecal Consistency

| | | | | | | | 5-ASA | | | Emoxypine | | | Ifenprodil | | | Radiprodil | | |
|---|---|---|---|---|---|---|---|---|---|---|---|---|---|---|---|---|---|---|
| | Naïve | | | TNBS-Vehicle | | | (100 mg/kg) | | | (160 mg/kg) | | | (30 mg/kg) | | | (30 mg/kg) | | |
| Days | Mean | SEM | N | Mean | SEM | N | Mean | SEM | N | Mean | SEM | N | Mean | SEM | N | Mean | SEM | N |
| 2. | 0.40 | 0.22 | 10 | 1.33 | 0.37 | 15 | 1.47 | 0.36 | 15 | 1.07 | 0.33 | 15 | 1.00 | 0.3900 | 15 | 1.20 | 0.43 | 15 |
| 3. | 0.60 | 0.25 | 10 | 2.15 | 0.39 | 15 | 1.60 | 0.45 | 15 | 1.73 | 0.47 | 15 | 1.71 | 0.4500 | 15 | 1.33 | 0.46 | 15 |
| 4. | 0.60 | 0.25 | 10 | 2.83 | 0.41 | 15 | 1.71 | 0.49 | 15 | 2.14 | 0.47 | 15 | 2.00 | 0.4700 | 15 | 1.23 | 0.45 | 15 |
| 5. | 0.40 | 0.22 | 10 | 2.55 | 0.47 | 15 | 1.38 | 0.44 | 15 | 1.71 | 0.45 | 15 | 1.67 | 0.4300 | 15 | 1.00 | 0.41 | 15 |
| 6. | 0.20 | 0.22 | 10 | 1.78 | 0.48 | 15 | 0.83 | 0.35 | 15 | 1.38 | 0.39 | 15 | 1.33 | 0.4000 | 15 | 1.00 | 0.41 | 15 |
| 7. | 0.20 | 0.16 | 10 | 1.56 | 0.43 | 15 | 0.83 | 0.35 | 15 | 0.92 | 0.34 | 15 | 1.17 | 0.4100 | 15 | 1.00 | 0.41 | 15 |

TABLE 6
Occult Positivity

| | | | | | | | 5-ASA | | | Emoxypine | | | Ifenprodil | | | Radiprodil | | |
|---|---|---|---|---|---|---|---|---|---|---|---|---|---|---|---|---|---|---|
| | Naïve | | | TNBS-Vehicle | | | (100 mg/kg) | | | (160 mg/kg) | | | (30 mg/kg) | | | (30 mg/kg) | | |
| Days | Mean | SEM | N | Mean | SEM | N | Mean | SEM | N | Mean | SEM | N | Mean | SEM | N | Mean | SEM | N |
| 2. | 0.00 | 0.00 | 10 | 1.73 | 0.33 | 15 | 1.33 | 0.37 | 15 | 1.33 | 0.32 | 15 | 1.86 | 0.3200 | 15 | 2.00 | 0.34 | 15 |
| 3. | 0.00 | 0.00 | 10 | 2.77 | 0.34 | 15 | 2.00 | 0.39 | 15 | 1.73 | 0.43 | 15 | 2.14 | 0.3800 | 15 | 2.53 | 0.36 | 15 |
| 4. | 0.00 | 0.00 | 10 | 3.50 | 0.23 | 15 | 2.43 | 0.41 | 15 | 2.43 | 0.36 | 15 | 2.62 | 0.3300 | 15 | 2.46 | 0.37 | 15 |
| 5. | 0.00 | 0.00 | 10 | 3.09 | 0.27 | 15 | 2.15 | 0.39 | 15 | 2.14 | 0.36 | 15 | 2.33 | 0.3000 | 15 | 2.33 | 0.37 | 15 |
| 6. | 0.00 | 0.00 | 10 | 2.67 | 0.26 | 15 | 1.67 | 0.30 | 15 | 1.85 | 0.25 | 15 | 2.00 | 0.3100 | 15 | 2.17 | 0.35 | 15 |
| 7. | 0.00 | 0.00 | 10 | 2.67 | 0.26 | 15 | 1.17 | 0.27 | 15 | 1.38 | 0.25 | 15 | 1.83 | 0.3500 | 15 | 1.83 | 0.35 | 15 |

TABLE 7
Colon Length, cm

| | | | | | | 5-ASA | | | Emoxypine | | | Ifenprodil | | | Radiprodil | | |
|---|---|---|---|---|---|---|---|---|---|---|---|---|---|---|---|---|---|
| Naïve | | | TNBS-Vehicle | | | (100 mg/kg) | | | (160 mg/kg) | | | (30 mg/kg) | | | (30 mg/kg) | | |
| Mean | SEM | N | Mean | SEM | N | Mean | SEM | N | Mean | SEM | N | Mean | SEM | N | Mean | SEM | N |
| 9.29 | 0.18 | 10 | 7.10 | 0.26 | 15 | 9.08 | 0.13 | 15 | 9.05 | 0.15 | 15 | 8.93 | 0.19 | 15 | 8.42 | 0.24 | 15 |

TABLE 8
Colon Weight, g

| | | | | | | 5-ASA | | | Emoxypine | | | Ifenprodil | | | Radiprodil | | |
|---|---|---|---|---|---|---|---|---|---|---|---|---|---|---|---|---|---|
| Naïve | | | TNBS-Vehicle | | | (100 mg/kg) | | | (160 mg/kg) | | | (30 mg/kg) | | | (30 mg/kg) | | |
| Mean | SEM | N | Mean | SEM | N | Mean | SEM | N | Mean | SEM | N | Mean | SEM | N | Mean | SEM | N |
| 0.1877 | 0.0046 | 10 | 0.2975 | 0.0193 | 15 | 0.2494 | 0.0196 | 15 | 0.2127 | 0.0116 | 15 | 0.2145 | 0.0156 | 15 | 0.3095 | 0.0317 | 15 |

TABLE 9
Colon Weight/Length ratio, mg/cm

| | | | | | | 5-ASA | | | Emoxypine | | | Ifenprodil | | | Radiprodil | | |
|---|---|---|---|---|---|---|---|---|---|---|---|---|---|---|---|---|---|
| Naïve | | | TNBS-Vehicle | | | (100 mg/kg) | | | (160 mg/kg) | | | (30 mg/kg) | | | (30 mg/kg) | | |
| Mean | SEM | N | Mean | SEM | N | Mean | SEM | N | Mean | SEM | N | Mean | SEM | N | Mean | SEM | N |
| 20.2228 | 0.3897 | 10 | 43.6037 | 4.2977 | 15 | 27.7482 | 2.4879 | 15 | 23.4383 | 1.0615 | 15 | 24.5065 | 2.3977 | 15 | 37.7622 | 4.5607 | 15 |

TABLE 10
Percent Survivability

| | | | 5-ASA | Emoxypine | Ifenprodil | Radiprodil |
|---|---|---|---|---|---|---|
| | | TNBS- | (100 | (160 | (30 | (30 |
| Days | Naïve | Vehicle | mg/kg) | mg/kg) | mg/kg) | mg/kg) |
| 1. | 100. | 100.000 | 100.000 | 100.000 | 100.000 | 100.000 |
| 2. | 100. | 100.000 | 100.000 | 100.000 | 93.000 | 100.000 |
| 3. | 100. | 86.670 | 100.000 | 100.000 | 93.000 | 100.000 |
| 4. | 100. | 80.000 | 93.000 | 93.000 | 86.600 | 86.670 |
| 5. | 100. | 73.000 | 86.670 | 93.000 | 80.000 | 80.000 |
| 6. | 100. | 60.000 | 80.000 | 86.670 | 80.000 | 80.000 |
| 7. | 100. | 60.000 | 80.000 | 86.670 | 80.000 | 80.000 |

TABLE 11
Histopathological severity score

| | | | | | | 5-ASA | | | Emoxypine | | | Ifenprodil | | | Radiprodil | | |
|---|---|---|---|---|---|---|---|---|---|---|---|---|---|---|---|---|---|
| Naïve | | | TNBS-Vehicle | | | (100 mg/kg) | | | (160 mg/kg) | | | (30 mg/kg) | | | (30 mg/kg) | | |
| Mean | SEM | N | Mean | SEM | N | Mean | SEM | N | Mean | SEM | N | Mean | SEM | N | Mean | SEM | N |
| 0.00 | 0.00 | 10 | 1.93 | 0.17 | 9 | 1.08 | 0.20 | 12 | 0.64 | 0.12 | 13 | 1.06 | 0.13 | 12 | 1.61 | 0.23 | 12 |

1-26. (canceled)

27. A method for the treatment or prophylaxis of colitis or inflammatory bowel disease (IBD) in a subject, the method comprising:
administering a therapeutically effective amount of Emoxypine to the subject.

28. The method of claim 27, wherein between 0.5 to 50 mg of Emoxypine per kg of the subject is administered to the subject.

29. The method of claim 28, wherein between 1 to 30 mg of Emoxypine per kg of the subject is administered to the subject.

30. The method of claim 29, wherein between 5 to 20 mg of Emoxypine per kg of the subject is administered to the subject.

31. The method of claim 30, wherein between 10 to 15 mg of Emoxypine per kg of the subject is administered to the subject.

32. The method of claim 30, wherein about 13 mg of Emoxypine per kg of the subject is administered to the subject.

33-46. (canceled)

47. The method of claim 27, further comprising administering one or more of the following: anti-inflammatory drugs, immune system suppressors, antibiotics, anti-diarrheals, pain relievers, iron supplements, vitamin B-12 shots, and calcium and vitamin D supplements.

48. The method of claim 47, wherein the anti-inflammatory drugs comprise one or more of the following: corticosteroids and oral 5-aminosalicylates.

49. The method of claim 47, wherein the immune system suppressors comprise one or more of the following: azathioprine, mercaptopurine, infliximab, adalimumab, certolizumab pegol and vedolizumab.

50. The method of claim 47, wherein the antibiotics comprise one or more of the following: ciprofloxacin and metronidazole.

51. The method of claim 27, wherein the inflammatory bowel disease is Crohn's disease.

52. The method of claim 27, wherein the inflammatory bowel disease is ulcerative colitis.
